(19) United States
(12) Patent Application Publication
(10) Pub. No.: US 2005/0214230 A1
(43) Pub. Date: Sep. 29, 2005

(54) Novel stomatological gel

(75) Inventors: Bharat Pravinchandra Mehta, Maharashtra (IN); Rajen Shah, Maharashtra (IN); Madhukant Mansukhlal Doshi, Maharashtra (IN)

Correspondence Address: LACKENBACH SIEGEL, LLP, LACKENBACH SIEGEL BUILDING, 1 CHASE ROAD, SCARSDALE, NY, 10583, US

(73) Assignee: J. B. Chemicals & Pharmaceuticals Ltd., Worli Bombay (IN)

(21) Appl. No.: 11/087,266

(22) Filed: Mar. 23, 2005

(30) Foreign Application Priority Data
Mar. 23, 2004 (RU) 2004108224
Mar. 22, 2005 (IN) 314/MUM/2005

(51) Int. Cl. A61K 7/16

(52) U.S. Cl. 424/49

(57) ABSTRACT

A novel pharmaceutical dental composition including diclofenac and chlorhexidine gluconate intended for pain associated with various diseases, comprising the diclofenac in its salt form and the chlorhexidine, and in addition a mucoadhesive agent in a pharmaceutically acceptable vehicle providing for bio-adhesion of the composition. One or more local anesthetics such a lidocaine and benzocaine, etc. may also optionally be included.

The invention relates to a novel pharmaceutical dental composition comprising of diclofenac and chlorhexidine gluconate intended for the treatment of pain associated in various dental diseases. The said composition employs a muco-adhesive agent providing for bio-adhesion of the composition thus exhibiting longer duration of action. Optionally the formulations may additionally contain one or more local anesthetics like lidocaine, benzocaine etc.

### BACKGROUND OF THE INVENTION

1. Field Of The Invention

The present invention relates to a novel pharmaceutical dental formulation for topical application of Diclofenac intended to alleviate pain associated with various dental diseases including periodontitis and Chlorhexidine Gluconate as a broad spectrum anti-microbial agent, the said formulation employing a muco-adhesive agent facilitating bio adhesion of the gel in periodontal pockets thereby retaining the drug at required site for longer duration and hence increasing the efficacy of the product.

2. Description Of The Prior Art

Periodontal disease is an inflammatory disease of the gums and has been a major concern in dentistry. The microorganisms most widely encountered are anaerobes and facultative streptococci. Periodontal disease encompasses specific conditions affecting the gingiva and the supporting connective tissues and alveolar bone. Gingivitis is thought to be caused by a non-specific bacterial plaque flora that gradually changes from predominantly gram positive to more gram negative. Periodontitis is accompanied by inflammation at least initially and pain and hence it is advisable to use one or more medication exhibiting anti-inflammatory and analgesic effects in course of the medicamental treatment.

The treatment of periodontal disease includes long acting capsules or tablets held in the mouth, buccal implants for releasing drugs into the saliva, topically applied gels, and topically applied drug containing bandages, impregnated or drug releasing forms of dental floss and solid absorbable fibers of polyglycolic acid with therapeutic agents incorporated therein.

In case of site-specific drug delivery for treatment of the periodontal pockets it is necessary to ensure retention of the drug near to the affected areas surrounding the teeth so as to facilitate diffusion of the therapeutic agent to the affected site.

Anti-inflammatory preparations include the medications that have an immediate effect upon different phases of inflammation. The inhibition of prostaglandin synthesis by Non-Steroidal Anti-Inflammatory drugs can alleviate the pain and inflammation associated with a variety of disorders.

Diclofenac is a Non-Steroidal Anti-Inflammatory drug presenting significant analgesic, anti-inflammatory and antipyretic properties. Chemically it is a phenylacetic acid derivative. It is a potent inhibitor of cyclo-oxygenase activity and causes sharp reduction in the formation of prostaglandin, prostacyclin and thromboxane product, all of which are mediators of inflammation. In addition diclofenac also regulates the lipoxygenase pathway.

The long-term administration of oral Diclofenac gives rise to side effects such as epigastric pain, nausea, vomiting and diarhhoea. Thus, to avoid the drawbacks of systemic administration, a dental gel for topical application of Diclofenac is desirable in periodontitis and other dental diseases.

Topical gel formulations with Diclofenac as the active ingredient have been known. They have however found application for formulations intended for percutaneous absorption or absorption via the skin.

Chlorhexidine is a bis biguanide antiseptic and disinfectant effective against a wide range of bacteria, some fungi and viruses. It shows rapid and persistent antimicrobial activity. It is a broad-spectrum antimicrobial agent effective against gram positive and gram negative bacteria.

A dental gel comprising of Chlorhexidine is also used for gingivitis and prevention of plaque. A dental composition containing Chlorhexidine Gluconate in various strengths of 0.1% to 1% in the form of topical application is also used for periodontal diseases (Br. Dental J., 1977,142,366-369). Chlorhexidine Gluconate 1% dental gel and 0.2% mouthwash is employed for the prevention of plaque and the prevention and treatment of gingivitis and in the treatment of oral candidiasis. U.S. Pat. No. 5,958,381 discloses antiplaque dentifrice employing Chlorhexidine along with an abrasive agent. Formulations containing Metronidazole Benzoate and Chlorhexidine have been described in U.S. Pat. No. 6,017,516 assigned to Lekar Pharma Ltd.

Mucoadhesive agents have been widely used due to their ability to retain the pharmaceutically active agents for extended period of time on any mucosal epithelia including those of eye, nose, mouth, rectum or vagina. These polymers first achieve intimate contact with the mucus and interpenetrate with the mucus to be retained at the site of application. Once the surfaces are in intimate contact, the adhesive polymer and the mucin glycoprotiens interdiffuse causing chain entanglement and bond formation and thus leading to retention at the site.

U.S. Pat. No. 5,350,769 describes an anti-inflammatory gel preparation comprising salts of diclofenac, a non-ionic polymer, a dibasic ester and a lower alcohol, the said preparation exhibiting superior percutaneous absorptivity and also high stability.

U.S. Pat. No. 5,939,047 relates to a composition and a method of treatment of periodontal and other related diseases employing one or more therapeutic agent and hyaluronic acid or its derivatives as a carrier. However, Hyaluronic acid being a substance of natural origin poses risk of microbial contamination. Besides high cost of hyaluronic acid gives rise to an expensive product.

U.S. Pat. No. 4,670,254 assigned to Toko Yakuhin Industry Co. Ltd. relates to an anti-inflammatory painless topical gel preparation of diclofenac with good stability characters comprising of diclofenac sodium, hydrophilic polymer of acrylic acid, an aliphatic amine and a solvent. The preparation so formulated is basically meant for topical application only.

U.S. Pat. No. 5,422,102 describes an anti-inflammatory and analgesic gel preparation comprising diclofenac, an ester of dibasic acid, a lower alcohol and a non-ionic polymer. The said preparation is claimed to exhibit superior medical effects. However it does not find any use in the treatment of dental diseases and has been specifically formulated for percutaneous absorption.

U.S. Pat. No. 6,471,970 relates to a method of treating periodontitis by applying a fluid pharmaceutical composition with controlled release of an active substance, the composition having the property of gelling instantaneously in presence of the aqueous phase. The composition comprises of a therapeutically active substance, a phospholipid, a fatty acid and a solvent.

U.S. Pat. No. 5,876,744 describes compositions having high bioadhesion and mucoadhesion containing mixtures of synthetic polymers like polyvinyl alcohol and polycarbophil and of biopolymers such as alginic acid hyaluronic acid and dermatan sulphate, the preparation meant for percutaneous absorption.

The prior art mentioned above presents a need to formulate an analgesic dental preparation exhibiting good mucoadhesive properties so as to retain the drug for a longer period of time in the affected area and cause local delivery of drug for therapeutic action.

The present invention relates to a novel gel composition suitable for dental application employing Diclofenac as the active ingredient and capable of delivering the active ingredient to the affected area with good retention characteristics so as to cause drug delivery over an extended period of time for the treatment of pain associated with dental diseases including periodontitis and additionally comprising Chlorhexidine Gluconate for its antimicrobial activity.

### SUMMARY OF THE INVENTION

It is an object of this invention to provide novel gel compositions for dental application capable of delivering the active ingredient to the affected area with good retention characteristics and suitable for the treatment of pain associated with various dental diseases including periodontitis. It is another object of the invention to provide novel gels for dental application comprising active ingredients Diclofenac and its salts as anti-inflammatory agent and Chlorhexidene gluconate as antimicrobial agent.

It is also an object of this invention to provide such compositions, which comprise mucoadhesive agents to render good retention characteristics.

Another object of the invention is to provide gel compositions for dental application that may further comprise local anaesthetic agents.

Other features, advantages and objectives of this invention and its preferred embodiments will become apparent from the detailed description and accompanying claims, which follow.

### DESCRIPTION OF THE PREFERRED EMBODIMENTS

The present invention relates to the pharmaceutical mucoadhesive dental gel formulation and manufacturing process thereof for topical application in the form of aqueous gel suitable for the treatment of pain associated with various dental diseases including periodontitis. The present invention more particularly relates to dental preparations containing Diclofenac and its salts in a concentration ranging form 1-5% as the active ingredient in a suitable gel formulation, a mucoadhesive agent to render good retention characteristics and containing Chlorhexidine Gluconate as an antimicrobial agent.

The active ingredient, diclofenac may be present in any of its salt forms including sodium, potassium and diethyl amine in a concentration ranging from 1-5%, the most preferred concentration being 3%.

The composition according to the present invention comprises of one or more antimicrobial agents selected from Benzyl alcohol, Benzalkonium Chloride, Cimitedine and Chlorhexidine Gluconate. The preferred antimicrobial agent is a Chlorhexidine Gluconate in a concentration ranging from 0.05 to 1%, more preferably 0.25% weight based on the total weight respectively of the said composition.

As mentioned above, the mucosal adhesive dental gel of this invention contains a mucoadhesive polymer selected from natural gums like tragacanth, sodium alginate, gelatin, karaya etc., pectin, chitosan, starch, modified celluloses, crystalline cellulose, microcrystalline cellulose, carboxymethyl cellulose, acrylic acid copolymer, copolymer of methyl vinyl ether and maleic anhydride, polyglycolic acid, polycarbophil A, the most preferred being copolymer of methyl vinyl ether and maleic anhydride. The said polymer is present in the range of 1.0-20% by weight based on the total weight of the composition, preferably about 1-10% and most preferred concentration being 2 % by weight based on the total weight of the composition.

Mucoadhesivity of the composition as described in the present invention can be attributed to interaction of gel layer of copolymer of methyl vinyl ether and maleic anhydride and mucin on the contact surface. These co-polymers are water soluble giving clear, tacky solutions with a solution rheology that can be modified by the addition of salts and bases. The dosage form as described in the present invention when applied to periodontal pockets, comes in contact with gingival cravicular fluid or saliva, the mucoadhesive polymer swells, giving rise to an increase in volume which facilitates maximum contact with mucin, the glycoprotein predominant in mucous layer, thus, increasing the contact time at the desired site and releasing the drugs slowly for longer duration. Adhesion to mucosal surface improves bioavailability and thus makes the product more efficacious.

Optionally the formulation according to the present invention may employ one or more local anesthetic agents selected from lignocaine, benzocaine, etodocaine and the like. The concentration of local anesthetic, especially lidocaine may range between 0.5 & 2 weight % in terms of lidocaine hydrochloride and the concentration of benzocaine as a local anesthetic may vary in the range of 1 to 20% and preferred concentration is 7.5%.

The composition according to the present invention employs a pharmaceutical acceptable vehicle comprising of water in a concentration ranging from 0-80% and one or more anhydrous solvents selected from glycerol, short chain ethers or esters, polyglycols, sorbital ethers and esters or polyethoxylated semisynthetic glycerides in a concentration ranging from 20-100%.

One of the preferred vehicles is a mixture of comprising water and propylene glycol. Propylene glycol concentration fluctuates between 5 to 80%, the preferred concentration being 20% by weight based on the total weight of the said composition.

Another preferred embodiment of the invention contains a mixture of PEG, more preferably PEG 400, diethylene glycol monoethyl ether, propylene glycol and glycerol.

The carboxyvinyl polymer used, as the gelling agent in the present invention is a hydrophilic polymer obtained by the polymerization of acrylic acid as the principal component. Preferred molecular weight of the polymer is in the range of 4×10⁶. Polymer present in the composition is in the range of 0.2 to 7% by weight based on the total weight of the said composition. Preferred polymer is carbomer 940 in said gelling agent in the present invention is selected from carbomer 940, carbomer 934, Hypromellose, sodium carboxymethylcellulose.

If the pH of the gel formulation of the present invention is on considerably acidic or basic side then it is desirable to add the pH modifier to the preparation of the present invention to adjust its pH in the range of 4.5-7.5, preferably 6 to 7. There are no specific limitations as to the kind of the pH modifiers are inorganic pH modifier, e.g. sodium hydroxide or potassium hydroxide. Preferred pH modifier in the present invention is sodium hydroxide solution.

Auxiliary Agents like chelating agents, sweeteners and flavouring agents can also be added to the gel preparations. Chelating agent used in this specification refers to disodium EDTA, Edetic acid, citric acid, Disodium calcium EDTA. Flavouring agents, which impart soothing action, refer to menthol, peppermint oil, spearmint oil, clove oil. Sweetening agent here refers to Saccharin sodium, aspartame, Dihydrochalcones, D-tryptophan etc.

Method of Preparation

The pharmaceutical gel composition according to the present invention can be prepared as follows: Diclofenac salt is first dissolved in propylene glycol. To the above solution, carboxy vinyl polymer and copolymer of methyl vinyl ether and maleic anhydride are added in portions. The solution so prepared is added to the solvent system with continuous stirring in homogenizer to form a gel. To the gel obtained, an aqueous solution of disodium EDTA, sodium saccharin and chlorhexidine gluconate is added with stirring till it dissolves. The pH of the resulting gel is then adjusted to about 6-7 with sodium hydroxide solution.

The present invention will now be further illustrated by, but is by no means limited to, the following examples wherein preferred embodiments of diclofenac containing dental gel preparations are expressed on the weight basis. Those who are skilled at the art can decide the percentage of other/auxiliary agents used to formulate the different example described below.

The following examples are provided to illustrate the present invention and should not be misunderstood to limit the scope of the present invention in any way.

### EXAMPLES

### Example 1

| | | QUANTITY |
|---|---|---|
| Sr No. | INGREDIENTS | (% w/w) |
| 1 | Diclofenac Sodium | 3 |
| 2 | Chlorhexidine Gluconate | 0.25 |
| 3 | Diethylene glycol monoethyl ether (Transcutol P) | 42.75 |
| 4 | Propylene glycol | 26 |
| 5 | Glycerol | 10 |
| 6 | Carbomer 934 | 3 |
| 7 | Gantrez S-97 BF | 2 |
| 8 | Aspartame | 1 |
| 9 | Sorbitol | 10 |
| 10 | Xylitol | 2 |
| 11 | Flavour | qs |
| | Total | 100 |

Manufacturing Procedure

1. Dissolve Diclofenac sodium in a mixture of anhydrous solvents consisting of propylene glycol+Diethylene glycol monoethyl ether (Transcutol P) under stirring forming vortex and add chlorhexidine gluconate to the solution.

2. Add aspartame, xylitol in step 1 under stirring forming vortex. Stir to dissolve.

3. Add sorbitol & glycerol to step 2 under stirring.

4. Disperse the following ingredients under stirring to above step 3.
a. Gantrez S-97
b. Carbomer 934

5. Heat the content to 60° C. & keep overnight.

6. Add flavor & mix well. Avoid air entrapment.

### Example 2

| | | QUANTITY |
|---|---|---|
| Sr No. | INGREDIENTS | (% w/w) |
| 1 | Diclofenac Sodium | 3 |
| 2 | Chlorhexidine gluconate | 0.25 |
| 3 | Diethylene glycol monoethyl ether (Transcutol P) | 54.9 |
| 4 | Propylene glycol | 26.1 |
| 5 | Glycerol | 10 |
| 6 | Carbomer 934 | 3 |
| 7 | Gantrez S-97 BF | 2 |
| 8 | Aspartame | 1 |
| 9 | Flavour | qs |
| | Total | 100 |

Manufacturing Procedure

1. Dissolve Diclofenac sodium in a mixture of anhydrous solvents consisting of propylene glycol+Diethylene glycol monoethyl ether (Transcutol P) under stirring and add chlorhexidine gluconate to the above solution.

2. Add aspartame, in step 1 under stirring. Stir to dissolve.

3. Add glycerol to step 2 under stirring.

4. Disperse the following ingredients under stirring to above step 3.
a. Gantrez S-97
b. Carbomer 934
c. Mix for 5 minutes.

5. Heat the content to 60° C. & keep overnight.

6. Add flavor & mix well. Avoid air entrapment.

### Example 3

| | | QUANTITY |
|---|---|---|
| Sr No. | INGREDIENTS | (% w/w) |
| 1. | Diclofenac Sodium | 3 |
| 2. | Benzocaine | 10 |
| 3. | Propylene glycol | 24 |
| 4. | Sodium metabisulphite | 0.3 |
| 5. | Sodium saccharine | 0.1 |
| 6. | HPMC K4M | 0.5 |
| 7. | Carbomer 940 | 2.5 |
| 8. | Gantrez S 97 | 2 |
| 9. | Disodium EDTA | 0.025 |
| 10. | Chlorhexidine gluconate | 0.05 |
| 11. | NaOH(10%) | qs to adjust pH (6.5) |
| 12. | Flavour | qs |
| | Water to make | 100 |

The gel preparations of the invention can be prepared for example, by initially dissolving diclofenac sodium in propylene glycol. Add carboxyvinyl polymer (carbomer 940), HPMC and copolymer of methyl vinyl ether and maleic anhydride ( Gantrez S 97) in portion with continuous stirring in homoginizer to form gel. To the gel thus obtained is added a separately prepared aqueous solution of disodium EDTA, sodium saccharin, sodium metabisulphite and chlorhexidine gluconate with stirring till it dissolves. Further, sodium hydroxide, pH modifier, is added to the resulting gel preparation, with stirring, in an amount sufficient to adjust the pH to about 5 to 6 which will result in a uniform viscous gel.

Clinical Trials

I. Clinical Evaluation of Short Term Efficacy of Combination of Diclofenac Sodium 1% and Chlorhexidine 0.25% in the Management of Gingivitis/Periodontitis

This study was undertaken to assess the efficacy of Diclofenac sodium 1%+Chlorhexidine 0.25% gel in reducing signs of inflammation in gingivitis and periodontitis and to see its effect on resolution of inflammation.

Materials And Methods: 60 Patients with Gingivitis/Early periodontitis in more than 6 teeth were included in the study. They were distributed into two groups. Group A (n=30) received (Diclofenac sodium 1%+Chlorhexidine 0.25% gel) with scaling. Group B (n=30) received no medication with scaling. The clinical parameters evaluated were Plaque Index (Loe & Silness), Gingival Index (Loe & Silness) and Pain (Visual analogue scale).

Results And Discussion: The percentage decreases in Plaque Index was maximum in Group A (61.9%) compared to Group B (36.3%) at the end of 4 weeks. Similarly, Gingival Index reduction was maximum in Group A (61.5%) compared to Group B (33.3%) at the end of 4 weeks. Pain Index reduction was maximum in Group A (83.33%) compared to Group B (20%) at the end of 4 weeks. The drug was well tolerated.

Conclusion:

1. Diclofenac sodium 1%+Chlorhexidine 0.25% brings about faster resolution as compared to just scaling.

2. Diclofenac sodium 1%+Chlorhexidine 0.25% gel is effective in the reducing signs and symptoms of inflammation associated with gingivitis and early periodontitis.

3. Diclofenac sodium 1%+Chlorhexidine 0.25% gel is a short term treatment with no side effect

II. Evaluation of Efficacy of Diclofenac Sodium 1% and Chlorhexidine 0.25% Gel for Topical Application in the Management of Gingivitis/Periodontitis

The aim of this study was to assess the efficacy of Diclofenac sodium 1%+Chlorhexidine 0.25% gel in reducing signs of inflammation in gingivitis and periodontitis.

Materials And Methods:50 Patients with Gingivitis/Early periodontitis in more than 6 teeth were included in the study. They were divided into two groups:
Group A (n=25) received (Diclofenac sodium 1%+Chlorhexidine 0.25% gel) with scaling.
Group B (n=25) received placebo gel with scaling.

Results: The clinical parameters evaluated were Plaque Index (Loe & Silness), Gingival Index (Loe & Silness) and Pain (Visual analogue scale).

The percentage decreases in average Plaque Index was maximum in Group A (75%) compared to Group B (40%) at the end of 4 weeks. Similarly, reduction in average Gingival Index was maximum in Group A (66.7%) compared to Group B (28%) at the end of 4 weeks. Average Pain Index reduction was 80% in Group A at the end of 4 weeks. Average Pain Index reduction was 25% in Group B.

Conclusion: Diclofenac sodium 1%+Chlorhexidine 0.25% gel for topical application was found to be very effective in patients of gingivitis/periodontitis. This preparation was well tolerated.

It is to be understood that the example and embodiments described hereinabove are for the purpose of providing a description of the present invention by way of example and are not to be viewed as limiting the present invention in any way. Various modifications or changes that may be made to that described hereinabove by those of ordinary skill in the art are also contemplated by the present invention and are to be included within the spirit and purview of this application and the following claims.

1. A novel dental gel composition comprising a therapeutic active amount of diclofenac in its salt form, Chlorhexidine gluconate and a mucoadhesive agent in a pharmaceutically acceptable vehicle.

2. A composition in accordance with claim 1, wherein the concentration of diclofenac is 1-5%, by weight based on the total weight of the said composition.

3. A composition according to claim 2, wherein the preferred concentration of diclofenac is 2-3%, by weight based on the total weight of the said composition.

4. A composition in accordance with claim 1, wherein the concentration of Chlorhexidine Gluconate ranges from 0.05-2% by weight based on the total weight of the said composition.

5. A composition in accordance with claim 1, wherein the concentration of Chlorhexidine Gluconate is 0.25% b weight based on the total weight of the said composition.

6. A composition in accordance with claim 1, wherein the said mucoadhesive agent is selected from the group consisting of natural gums like tragacanth, sodium alginate, gelatin, karaya etc., pectin chitosan, starch, modified celluloses, crystalline cellulose, microcrystalline cellulose, carboxymethyl cellulose, acrylic acid copolymer, copolymer of methyl vinyl ether and maleic anhydride, polyglycolic acid, polycarbophil A.

7. A composition in accordance with claim 6, wherein the mucoadhesive agent is a copolymer of methyl vinyl ether and maleic anhydride.

8. A composition in accordance with claim 6, wherein the said mucoadhesive polymer is present in a concentration ranging from 1-20%, by weight based on the total weight of the said composition.

9. A composition in accordance with claim 8, wherein the range of the said mucoadhesive polymer is between 1-10%, by weight based on the total weight of the said composition.

10. A composition in accordance with claim 9, wherein the concentration of said mucoadhesive polymer is 2%, by weight based on the total weight of the said composition.

11. A composition in accordance with claim 1, wherein the pharmaceutically acceptable vehicle comprises of water in a concentration ranging from 0-80% and one or more solvents selected from the group consisting of glycerol, short chain ethers or esters, polyglycols, sorbital ethers and esters or polyethoxylated semisynthetic glycerides.

12. A composition in accordance with claim 11, wherein one or more solvents are selected from the group consisting of propylene glycol, glycerin, polyethylene glycols and glycerol.

13. A composition in accordance with claim 12, wherein the said mixture containing Propylene Glycol, Diethylene glycol monoethyl ether and glycerol is in a ratio of 8:5:1.

14. A composition in accordance with claim 1, containing a gelling agent which is hydrophilic and a water dispersible polymer selected from the group consisting of carbomer 940, carbomer 934, hypromellose and sodium carboxymethylcellulose in the range of 0.2 to 7% by weight based on the total weight of said composition.

15. A composition in accordance with claim 14, wherein said gelling agent is carbomer 940 in an amount of about 1.5-5% by weight based on the total weight of the said composition.

16. A composition in accordance with claim 1, comprising a chelating agent selected from the group consisting of Disodium EDTA, Edetic acid, Citric acid and Disodium Calcium EDTA.

17. A composition in accordance with claim 16, wherein said chelating agent is Disodium EDTA, in the range of about 0.01% to about 0.1% by weight based on the total weight of said composition.

18. A composition in accordance with claim 1, comprising a sweetening agent selected from the group consisting of saccharin sodium, aspartame, dihydrochalcones and trytophan.

19. A composition in accordance with claim 18 wherein said sweetening agent is saccharin sodium.

20. A composition in accordance with claim 1, wherein the flavoring agent is selected from the group consisting of menthol, peppermint oil, spearmint oil, anise oil and clove oil.

21. A composition in accordance with claim 1, having a pH in the range of 4.5 to 7.5.

22. A composition in accordance with claim 1, comprising a local anesthetic selected from the group consisting of lidocaine and benzocaine or etidocaine in required concentrations.

23. A process for preparation of the composition in accordance with claim 1 consisting of the following steps:
a) dissolve Diclofenac in propylene glycol solvent;
b) add carboxy vinyl polymer and a copolymer of methyl vinyl either and maleic anhydride to the above solution;
c) add the solution so obtained in step b to a solvent system prepared separately with constant stirring with a homogenizer; and
d) to the gel obtained, add an aqueous solution of Disodium EDTA, sodium saccharin and chlorhexidine gluconate with stirring till it dissolves.
